(19) United States
(12) Patent Application Publication
(10) Pub. No.: US 2025/0134923 A1
(43) Pub. Date: May 1, 2025

(54) SUPERABSORBENT HYDROGELS WITH LIPASE INHIBITOR

(71) Applicant: JUNION LABS PTE. LTD., Singapore (SG)

(72) Inventors: Jingnan LUO, Singapore (SG); Hongqian BAO, Singapore (SG); Bo Huai Moses LEE, Singapore (SG)

(21) Appl. No.: 18/689,409

(22) PCT Filed: Sep. 5, 2022
(86) PCT No.: PCT/SG2022/050640

§ 371 Date: Mar. 5, 2024

Related U.S. Application Data
(60) Provisional application No. 63/261,026, filed on Sep. 9, 2021.

(51) Int. Cl. A61K 31/738; A61K 9/00; A61K 9/16; A61K 9/48; A61K 45/06; A61P 3/04

(52) U.S. Cl. CPC A61K 31/738; A61K 9/0053; A61K 9/16; A61K 9/48; A61K 45/06; A61P 3/04

(57) ABSTRACT

The present invention relates to a pharmaceutical composition which, when orally consumed by a patient is able to reduce food consumption of the patient via temporary gastric space occupation as well as reduce the patient's absorption of the consumed food via enzyme inhibition. The present invention comprises a hydrophilic polymer crosslinked with a crosslinker and a lipase inhibitor, wherein the crosslinked hydrophilic polymer has a media uptake ratio (MUR) of at least 20 and an elastic modulus (G′) value in the range of 100 Pa to 10,000 Pa, wherein the composition has a weight ratio of the crosslinked hydrophilic polymer to the lipase inhibitor which is greater than 10.

### TECHNICAL FIELD

The present invention relates to a composition which, when orally consumed by a patient is able to reduce food consumption of the patient via temporary gastric space occupation, as well as to reduce the patient's absorption of the consumed food via enzyme inhibition. The present invention comprises a hydrophilic polymer crosslinked with a crosslinker and a lipase inhibitor, wherein the crosslinked hydrophilic polymer has a media uptake ratio (MUR) of at least 20 and an elastic modulus (G′) value in the range of 100 Pa to 10,000 Pa, and wherein the composition has a weight ratio of the crosslinked hydrophilic polymer to the lipase inhibitor which is greater than 10. The present invention also relates to a method of forming the composition, a capsule comprising the composition, a method of treating obesity, pre-diabetes, diabetes, non-alcoholic fatty liver disease, non-alcoholic steatohepatitis, chronic idiopathic constipation, reducing caloric intake or improving glycemic control using the composition or the capsule and medical uses thereof.

### BACKGROUND ART

Obesity is a condition in which excess body fat has accumulated to such an extent that health may be negatively affected. Obesity is associated with various diseases, particularly non-alcoholic steatohepatitis (NASH), diabetes mellitus type 2, cardiovascular diseases, obstructive sleep apnea, certain types of cancer, and osteoarthritis. Non-alcoholic steatohepatitis (NASH) is an advanced form of non-alcoholic fatty liver disease (NAFLD). NAFLD is caused by build-up of fat in the liver. When this build-up causes inflammation and damage, it is known as NASH, which can lead to scarring of the liver, a potentially life-threatening condition called cirrhosis. Worldwide, both obesity and NASH have been on the rise and current conservative treatment modalities such as lifestyle modification and dietary orientation are broadly available, but unfortunately, are poorly effective.

One method used in promoting long-term weight loss for the treatment of obesity and NASH is bariatric surgery. Bariatric surgery makes changes to the digestive system by restricting how much food is consumed by the patient in one sitting or by reducing the nutrients absorbed by the patient or both. Bariatric surgery has been proven to be extremely efficient at promoting long-term weight loss and improvement of obesity-related diseases. For the majority of patients who have obesity and metabolic syndrome, bariatric surgery has been demonstrated to have a clear benefit in reducing all of the components of NASH, including steatosis, steatohepatitis, and fibrosis. There have also been studies conducted that show bariatric surgery was able to resolve NASH without worsening fibrosis in up to 84% of patients with evaluable biopsies. However, bariatric surgery also has drawbacks in the form of complications. Although the mortality rate of bariatric surgery is less than 1%, non-fatal adverse events are far more common, and depending on the study, the overall complication rate can be as high as 23% with a reoperation rate of up to 12%. Those risks, along with high costs, restricted access, and misinformation, restrain the range of action of bariatric surgery substantially.

Over the years, alternative treatments that are similar to bariatric surgery were developed for patients who are not amenable to or not willing to undergo bariatric surgery. One such group of alternative treatment is endoscopic bariatric therapies (EBTs). EBTs work similarly to bariatric surgery and include the use of intragastric balloons, gastric suturing and gastric plication, endoscopic magnetic anastomosis, aspiration therapy, intermittent gastric outlet obstruction, gastric/duodenal/jejunal bypass liners, and interventions in the small bowel focused not only on overweight but also on diabetic patients. Although EBTs are significantly less invasive than bariatric surgery and have proven to be effective in the treatment of obesity and its related diseases, it still requires the patient to undergo an invasive medical procedure which might cause some patients to reject it.

Therefore, there is a need for a novel, non-invasive way to treat obesity which leverages the effectiveness of bariatric surgery of restricting how much food is consumed by the patient in one sitting and restricting nutrients that are absorbed by the patient in order to treat or prevent obesity and NASH.

### SUMMARY

In an aspect, there is provided a composition comprising:
a lipase inhibitor; and
a hydrophilic polymer crosslinked with a crosslinker;
wherein the crosslinked hydrophilic polymer has a media uptake ratio (MUR) of at least 20, and an elastic modulus (G′) value in the range of 100 Pa to 10,000 Pa, and
wherein a weight ratio of the crosslinked hydrophilic polymer to the lipase inhibitor is greater than 10.

Advantageously, the composition, when orally consumed by a patient, may be able to reduce food consumption of the patient via temporary gastric space occupation as well as reduce the absorption of the consumed food by enzyme inhibition.

Advantageously, a synergistic effect in terms of treatment of obesity, pre-diabetes, diabetes, non-alcoholic fatty liver disease (NAFLD), non-alcoholic steatohepatitis (NASH), or chronic idiopathic constipation, or of reducing caloric intake or improving glycemic control may be observed with the composition as defined above, as the crosslinked hydrophilic polymer may reduce appetite and reduce consumption of food by swelling in the stomach after ingestion, and the lipase inhibitor may decrease dietary lipid absorption, thereby reducing the amount of calories absorbed by the body.

When the composition is orally consumed by a patient, the hydrophilic polymer may absorb water or gastric fluid in the stomach and swell. Since the swelled hydrophilic polymer particles may have a sufficiently high elastic modulus of greater than 20, they may form a solid mass within the stomach. Similar to an intragastric balloon, this may cause the patient to feel satiated and thus reduce food consumption. Therefore, it may be important for the hydrophilic polymer to have a high media uptake ratio (MUR) of at least 20, as this may allow a smaller amount of hydrophilic polymer to be needed to achieve the desired swelled size. However, unlike an intragastric balloon, the solid mass formed by the swelled hydrophilic particles may advantageously be able to be naturally expelled from the stomach. This may be possible because the solid mass may be made up of many individual particles which may naturally be expelled from the stomach.

In an example, the crosslinked hydrophilic polymer may be in the form of a powder having a particle size in the range of 0.01 mm to 5 mm. Advantageously, this particle size may facilitate the crosslinked hydrophilic polymer to be naturally expelled from the stomach.

Advantageously, the lipase inhibitor in the composition may be able to bind to lipase enzymes in the intestine, thus preventing the hydrolysis of dietary triglycerides into monoglycerides and fatty acids, thereby reducing the amount of calories absorbed by the body. Similar to gastric/duodenal/jejunal bypass liners, this may reduce the absorption of dietary fat by the patient.

Further advantageously, other than offering direct weight loss benefits, lipase inhibitors may also aid in the treatment of NASH. Lipase inhibitors may be able to reduce the serum levels of lipopolysaccharide, periostin and tumour necrosis factor-α, while increasing the levels of protective endocrine cytokines, such as adiponectin. These changes brought forth by the consumption of lipase inhibitors have been reported to promote the amelioration of NASH.

An example of a lipase inhibitor may be Orlistat. Orlistat, also known as tetrahydrolipstatin, is the saturated derivative of lipstatin, a potent natural inhibitor of pancreatic lipases isolated from the bacterium Streptomyces toxytricini. Its primary function may be to advantageously prevent the absorption of lipids present in the diet by acting as a lipase inhibitor, thereby reducing caloric intake. When administered with fat-containing foods, Orlistat may partially inhibit hydrolysis of triglycerides, thus reducing the subsequent absorption of monoaclglycerides and free fatty acids. When administered at therapeutic doses (120 mg with meals), the inhibition of fat absorption (approximately 30% of ingested fat) may contribute to an additional caloric deficit of approximately 200 calories.

Orlistat has advantageously been shown to treat and prevent type II diabetes mellitus by oral administration of 60-720 mg per day and effectively reduce haemoglobin A1c levels. However, a known adverse effect of Orlistat is that it may cause anal leakage of lipid due to the physical separation of unabsorbed dietary lipid from the bulk of the unabsorbable food solids while it passes through the lower large intestine. This may lead to gastrointestinal side-effects such as flatulus, fatty/oily stools, increased defecation, faecal urgency or faecal incontinence and abdominal pain, which may create considerable discomfort to the subject taking Orlistat. These side effects may be common to all lipase inhibitors.

The composition as defined above may advantageously mitigate the known side effects, such as anal leakage of lipid, associated with the use of lipase inhibitors. Without being bound to theory, advantageously, the oral co-administration of the crosslinked hydrophilic polymer with the lipase inhibitor to a patient may reduce food consumption of the patient as well as reduce the absorption of the consumed food of the patient, while also preventing the anal leakage of lipid and minimise the side-effects associated with lipase inhibitors.

In an example, the crosslinker of the crosslinked hydrophilic polymer may be a spacer crosslinker comprising a first optionally substituted aliphatic moiety terminated at each end with a second moiety comprising at least two carboxylic acid groups

Advantageously, where the crosslinker is the spacer crosslinker, the crosslinked hydrophilic polymer may form generally more stable and stiff networks compared to polymers that are associated by non-chemical, physical interactions only. Advantageously, the three-dimensional structure of the hydrogel that may be formed from the crosslinked hydrophilic polymer may be maintained in the stomach by using the spacer crosslinker, which may result in delayed emptying time. The spacer crosslinker may be first prepared by reacting a spacer having two or more hydroxyl groups, for example polyethylene glycol (PEG) with a molecule having two or more carboxylic acid groups, for example citric acid (CA). Thereafter, the spacer crosslinker may be reacted with a hydrophilic polymer having hydroxyl groups. Using a long hydrophilic crosslinker may allow for the formation of a hydrogel with a looser polymeric network, which in turn may have a higher swelling ratio, while still achieving a high level of mechanical strength, as measured by the swelled state elastic modulus.

Advantageously, the crosslinked hydrophilic polymer as defined above having the specified media uptake ratio (MUR), elastic modulus, and having the specified weight ratio of the crosslinked hydrophilic polymer to the lipase inhibitor, may facilitate better control over gastric space occupation, which may result in better performance of the composition in terms of gastric retention and emptying compared to previously known hydrogels that are randomly crosslinked.

In an example, the composition may further comprise an amylase inhibitor, a glucosidase inhibitor or any mixture thereof

Advantageously, the presence of an amylase inhibitor and/or glucosidase inhibitor in the composition may further reduce the absorption of dietary calories.

In another aspect, there is provided a method of forming the composition as defined above, comprising the step of contacting a lipase inhibitor with a hydrophilic polymer crosslinked with a crosslinker;
wherein the crosslinked hydrophilic polymer has a media uptake ratio (MUR) of at least 20, and an elastic modulus (G′) value in the range of 100 Pa to 10,000 Pa, and
wherein a weight ratio of the crosslinked hydrophilic polymer to the lipase inhibitor is greater than 10.

In another aspect, there is provided a capsule comprising the composition as defined above.

In another aspect, there is provided a method of treating obesity, pre-diabetes, diabetes, non-alcoholic fatty liver disease (NAFLD), non-alcoholic steatohepatitis (NASH), or chronic idiopathic constipation, or of reducing caloric intake or improving glycemic control in a subject in need thereof, comprising the step of administering to the subject a therapeutically effective amount of the composition as defined above or the capsule as defined above.

In another aspect, there is provided a composition as defined above or the capsule as defined above for use in the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

In another aspect, there is provided the use of the composition as defined above or the capsule as defined above in the manufacture of a medicament for the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

### Definitions

The following words and terms used herein shall have the meaning indicated:

“Alkyl” as a group or as part of a group refers to a straight or branched aliphatic hydrocarbon group, preferably a C₁-C₆ alkyl, unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkyloxy” refers to an alkyl group as defined herein that is singularly bonded to oxygen. The group may be a terminal group or a bridging group. If the group is a terminal group, it is bonded to the remainder of the molecule through the alkyl group.

“Heteroalkyl” refers to a straight- or branched-chain alkyl group preferably having from 2 to 6 carbons in the chain, one or more of which has been replaced by a heteroatom selected from S, O, P and N. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₁-C₁₂ heterocycloalkyl group. A heterocycloalkyl group may comprise 3 to 8 ring atoms. A heterocycloalkyl group may comprise 1 to 3 heteroatoms independently selected from the group consisting of N, O and S. The group may be a terminal group or a bridging group.

The term “optionally substituted” as used herein means the group to which this term refers may be unsubstituted, or may be substituted with one or more groups independently selected from acyl, alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, cycloamino, halo, carboxyl, haloalkyl, haloalkynyl, alkynyloxy, heteroalkyl, heteroalkenyl heteroalkynyl, heteroalkyloxy, hydroxyl, hydroxyalkyl, alkoxy, thioalkoxy, alkenyloxy, haloalkoxy, haloalkenyl, haloalkynyl, haloalkenyloxy, nitro, amino, nitroalkyl, nitroalkenyl, nitroalkynyl, nitroheterocyclyl, alkylamino, dialkylamino, alkenylamine, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as phosphono and phosphinyl, sulfinyl, sulfinylamino, sulfonyl, sulfonylamino, aryl, arylalkyl, arylalkyloxy, arylamino, Arylheteroalkyl, heteroaryl, heteroarylalkyl, heteroarylalkenyl, heteroarylalkynyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

There is provided a pharmaceutical composition comprising:
a lipase inhibitor; and
a hydrophilic polymer crosslinked with a crosslinker;
wherein the hydrophilic polymer has a media uptake ratio (MUR) of at least 20 and, and an elastic modulus (G′) value in the range of 100 Pa to 10,000 Pa, and
wherein a weight ratio of the crosslinked hydrophilic polymer to the lipase inhibitor is greater than 10.

The crosslinked hydrophilic polymer may be a hydrogel. The crosslinked hydrophilic polymer may be a super-absorbent polymer (SAP) hydrogel.

The hydrogel may be the crosslinked hydrophilic polymer further comprising a liquid. The hydrogel may be the crosslinked hydrophilic polymer that has been swelled in a liquid. The liquid may be an aqueous liquid. The liquid may be water, buffer, gastric fluid, simulated gastric fluid or any mixture thereof.

Hydrogels may be obtained by physical or chemical stabilization of aqueous solutions of polymeric fibre. Physical stabilization may be achieved via hydrogen bonds, hydrophobic interactions, and chain entanglements. These interactions may be generally reversible, and hence hydrogels resulting from crosslinked hydrophilic polymers that comprise mainly physical interactions may easily flow or degrade. In contrast, chemical crosslinks consist of covalent chemical bonds, and hydrogels formed using hydrophilic polymers comprising chemical crosslinks, may form generally more stable and stiff networks. The degree of crosslinking and type of crosslinker used may affect the physical properties of the resulting hydrogel, such as the degree of water retention, mechanical strength and degradation rate.

A hydrogel may be a network of crosslinked polymer chains that are hydrophilic and are able to absorb aqueous solutions through hydrogen bonding with water molecules. The water molecules may be retained within the hydrogel leading to the hydrogel swelling to multiple times its original volume in the process. The structural integrity of the hydrogel network may be maintained in water due to the crosslinking that holds the hydrophilic polymer chains together, forming a three dimensional solid. Superabsorbent polymer hydrogels (SAPs) are hydrogels which are able to absorb and retain extremely large amounts of a liquid relative to its own mass. In deionized and distilled water, a SAP may absorb 300 times its weight (from 30 to 60 times its own volume) and can become up to 99.9% liquid.

The total absorbency and swelling capacity of a hydrogel may be controlled by the type and degree of crosslinkers used to make the gel. For example, low-density crosslinked SAPs which have a tapped density less than about 0.1 g/mL may generally have a higher absorbent capacity and swell to a larger degree, resulting in a softer and stickier hydrogel formation. In contrast, SAPs with a high crosslinking density of higher than about 0.2 g/mL may exhibit a lower absorbent capacity and swelling, but the gel strength may be higher and its particle shape may be maintained even under modest pressure.

The hydrophilic polymer of the crosslinked hydrophilic polymer may be selected from the group consisting of polysaccharide, polyacrylate, polyacrylamide, polymer of ethylene maleic anhydride, polyvinyl alcohol, polyvinylpyrrolidone, crosslinked polyethylene oxide, starch grafted polyacrylonitrile, protein, glycoprotein, proteoglycan and any copolymer thereof.

The hydrophilic polymer of the crosslinked hydrophilic polymer may be a polysaccharide. The polysaccharide may be a compound selected from the group consisting of starch, hydroxyethyl starch, hydroxypropyl starch, carboxymethyl starch, amylose, dextran, chitin, pullulan, gellan gum, xylan, carrageenan, agar, locust bean gum, guar gum, gum arabic, pectin, cellulose, methylcellulose, ethylcellulose, hydroxyethylcellulose, hydroxypropyl-cellulose, ethylhydroxyethylcellulose, hydroxybutylmethylcellulose, hydroxyethylmethylcellulose, oxidized cellulose, carboxymethylcellulose, galactomannan, alginate, chitosan, cyclodextrin, xanthan, hyaluronic acid, heparin, chondroitin sulfate, keratan, dermatan, and polysaccharides having glycosamine residues in natural or diacetylated form and any mixture thereof. The polysaccharide may be a derivative of a compound selected from the group consisting of starch, hydroxyethyl starch, hydroxypropyl starch, carboxymethyl starch, amylose, dextran, chitin, pullulan, gellan gum, xylan, carrageenan, agar, locust bean gum, guar gum, gum arabic, pectin, cellulose, methylcellulose, ethylcellulose, hydroxyethylcellulose, hydroxypropyl-cellulose, ethylhydroxyethylcellulose, hydroxybutylmethylcellulose, hydroxyethylmethylcellulose, oxidized cellulose, carboxymethylcellulose, galactomannan, alginate, chitosan, cyclodextrin, xanthan, hyaluronic acid, heparin, chondroitin sulfate, keratan, dermatan, and polysaccharides having glycosamine residues in natural or diacetylated form and any mixture thereof.

Given the growing concern on environmental protection, recent interest has focused on the development of superabsorbent hydrogels based on biodegradable materials. Suitable biodegradable hydrophilic polymers include polysaccharides, such as alginate, starch, and cellulose derivatives. Polysaccharides may also be more biocompatible, such that they are safer when ingested.

The polysaccharide may comprise at least one carboxymethyl group.

The polysaccharide may be carboxylmethylcellulose.

Carboxymethylcellulose (CMC) or cellulose gum is a cellulose derivative with carboxymethyl groups (—CH₂—COOH) bound to some of the hydroxyl groups of the glucopyranose monomers that make up the cellulose backbone. CMC can be synthesized by the alkali-catalysed reaction of cellulose with chloroacetic acid. This reaction is followed by a purification process to produce pure CMC for use in food, pharmaceutical, and dentifrice (toothpaste) applications.

CMC can be used in food as a viscosity modifier or thickener, and to stabilize emulsions in various products including ice cream. It is also a constituent of many non-food products, such as toothpaste, laxatives, diet pills, water-based paints, detergents, textile sizing, reusable heat packs, and various paper products. It is used primarily because it has high viscosity, is nontoxic, and is generally considered to be hypoallergenic, since the major source of fibre is either softwood pulp or cotton linter.

The carboxymethylcellulose may have a degree of substitution in the range of about 0.6 to about 1.0, about 0.6 to about 0.8 or about 0.8 to about 1.0.

The functional properties of CMC may depend on the degree of substitution of the cellulose structure, as well as the chain length of the cellulose backbone structure and the degree of clustering of the carboxymethyl substituents. A degree of substitution in the range of about 0.6 to about 1.0 allows for better emulsifying properties and improves resistance to acids and salts.

The polysaccharide may have a viscosity, as a 1% (wt/wt) aqueous solution at 25° C., of greater than about 1000 cps, greater than about 2000 cps, greater than about 3000 cps, greater than about 5000 cps, greater than about 7000 cps, or greater than about 10,000 cps. The polysaccharide may have a viscosity, as a 1% (wt/wt) aqueous solution at 25° C., in the range of about 1000 cps to about 12000 cps, about 1000 cps to about 5000 cps, about 1000 cps to about 10,000 cps, about 5000 cps to about 10,000 cps, about 5,000 cps to about 12,000 cps or about 10,000 cps to about 12,000 cps.

The polysaccharide molecular weight may have a polydispersity index of less than 10, less than 5 or less than 2. The polysaccharide may have a polydispersity index, in the range of about 1 to about 10.

The crosslinking between the crosslinked hydrophilic polymer may be between the hydrophilic polymers directly or may be achieved via the use of a crosslinker.

The crosslinker of the crosslinked hydrophilic polymer may be the hydrophilic polymer itself. When the crosslinker of the crosslinked hydrophilic polymer is the hydrophilic polymer itself, the hydrophilic polymer may be considered self-crosslinked, whereby the whole hydrophilic polymer or a part of the hydrophilic polymer acts as the crosslinker. The hydrophilic polymer may comprise different segments that may each individually act as the crosslinker.

The crosslinker of the crosslinked hydrophilic polymer may be a multifunctional crosslinker. The crosslinker of the crosslinked hydrophilic polymer may be a bifunctional crosslinker or a trifunctional crosslinker.

The crosslinker of the crosslinked hydrophilic polymer may comprise at least two reactive groups selected independently from the group consisting of hydroxyl group, vinyl group, acrylic group, alkenyl group, alkynyl group, amino group, amido group, carboxylic acid group, ester group and any combination thereof.

The crosslinker of the crosslinked hydrophilic polymer may comprise at least two reactive groups independently selected from the group consisting of styrene, vinyl-toluene, vinyl ester of saturated C₁-C₄-carboxylic acid, alkyl vinyl ether with at least 2 carbon atoms in the alkyl group, acrylic and methacrylic ester, conjugated diolefin, allene, olefin halide, ethylene, propene, isobutylene, butadiene, isoprene, ester of monoethylenically unsaturated C₃-C₆-carboxylic acid, N-vinyllactam, acrylic and methacrylic ester of alkoxylated monohydric saturated alcohol, vinyl pyridine, vinyl morpholine, N-vinylformamide, dialkyldiallylammonium halide, N-vinylimidazol, N-vinylimidazoline, acrylamide, methacrylamide, acrylonitryl and any combination thereof.

The crosslinker of the crosslinked hydrophilic polymer may be selected from the group consisting of polyvinyl alcohol, methylene bis(acrylamide), polyethylene glycol, chitosan, bismaleimide, and any mixture thereof.

The crosslinker of the crosslinked hydrophilic polymer may comprise at least two carboxylic acid groups.

The crosslinker of the crosslinked hydrophilic polymer may be citric acid, oxalic acid, pyromellitic acid, butanetetracarboxylic acid, benzoquinonetetracarboxylic acid and any other mixture thereof.

The crosslinker of the crosslinked hydrophilic polymer may be a spacer crosslinker, wherein the spacer crosslinker comprises a first optionally substituted aliphatic moiety terminated at each end with a second moiety comprising at least two carboxylic acid groups.

The spacer crosslinker may have the following formula (I):

A-L-Z-L-A (I)
wherein
Z is the first optionally substituted aliphatic moiety;
A is the second moiety comprising at least two carboxylic acid groups; and
L is a linking group.

The first optionally substituted aliphatic moiety or Z may be derived from a first optionally substituted aliphatic molecule comprising at least two hydroxy groups. In this context, “derived” means that the first optionally substituted aliphatic moiety is formed as a result of the at least two hydroxyl groups of the first optionally substituted aliphatic molecule reacting with the second molecule as defined further below to form part of the linker L in formula (I).

The first optionally substituted aliphatic molecule may be a linear molecule and be terminated at each end with a hydroxyl group.

The first optionally substituted aliphatic molecule may have a molecular weight in the range of about 0.1 kDa to about 100 kDa, about 0.1 kDa to about 0.2 kDa, about 0.1 kDa to about 0.5 kDa, about 0.1 kDa to about 1 kDa, about 0.1 kDa to about 2 kDa, about 0.1 kDa to about 5 kDa, about 0.1 kDa to about 10 kDa, about 0.1 kDa to about 20 kDa, about 0.1 kDa to about 50 kDa, about 0.2 kDa to about 0.5 kDa, about 0.2 kDa to about 1 kDa, about 0.2 kDa to about 2 kDa, about 0.2 kDa to about 5 kDa, about 0.2 kDa to about 10 kDa, about 0.2 kDa to about 20 kDa, about 0.2 kDa to about 50 kDa, about 0.2 kDa to about 100 kDa, about 0.5 kDa to about 1 kDa, about 0.5 kDa to about 2 kDa, about 0.5 kDa to about 5 kDa, about 0.5 kDa to about 10 kDa, about 0.5 kDa to about 20 kDa, about 0.5 kDa to about 50 kDa, about 0.5 kDa to about 100 kDa, about 1 kDa to about 2 kDa, about 1 kDa to about 5 kDa, about 1 kDa to about 10 kDa, about 1 kDa to about 20 kDa, about 1 kDa to about 50 kDa, about 1 kDa to about 100 kDa, about 2 kDa to about 5 kDa, about 2 kDa to about 10 kDa, about 2 kDa to about 20 kDa, about 2 kDa to about 50 kDa, about 2 kDa to about 100 kDa, about 5 kDa to about 10 kDa, about 5 kDa to about 20 kDa, about 5 kDa to about 50 kDa, about 5 kDa to about 100 kDa, about 10 kDa to about 20 kDa, about 10 kDa to about 50 kDa, about 10 kDa to about 100 kDa, about 20 kDa to about 50 kDa, about 20 kDa to about 100 kDa, or about 50 kDa to about 100 kDa.

Using a long hydrophilic spacer crosslinker (such as one having the molecular weight as defined above) allows for the formation of a crosslinked hydrophilic polymer with a looser polymeric network while still achieving a high level of strength, as measured by the swelled state tensile modulus. A looser polymeric network leads to a greater swelling ratio of the hydrogel as it allows the polysaccharide chains within the network to move further away from each other, allowing for the polymer network to swell to a greater extent.

When a short crosslinker such as citric acid is used, two polysaccharide chains can be connected at a distance via third polysaccharide chain linking the two chains. However, the length of the connection is random. Therefore, generally, the linker length determines the proximity of the connected polysaccharide chains. Since multiple crosslinkers can be attached to a single chain at random points, using a short crosslinker results in a polymeric network in which the polysaccharide chains are linked close together, leading to a compacted network. In contrast, when long hydrophilic crosslinkers are used, the distance between two polysaccharide chains will be determined by the length of the long hydrophilic crosslinker. Since the polysaccharide chains will be connected to each other via a fixed chain length corresponding to the length of the long hydrophilic crosslinker, using a long hydrophilic crosslinker will lead to a looser polymeric network.

The strength of the hydrogel depends on the degree of interaction between the hydrophilic polymeric chains. When a short crosslinker such as citric acid is used, once one end of the crosslinker reacts with a polysaccharide chain, the other end can only react within the same polysaccharide chain, or with another polysaccharide chain that is in close proximity with the first polysaccharide chain, due to its short length. This severely limits the crosslinking network that can be formed. The mobility of a short crosslinker that has reacted on one end with a polysaccharide chain is low, as the polysaccharide chain is itself long and relatively immobile.

This limited mobility prevents the other end of the crosslinker to move around, and therefore results in crosslinks to form within the same polysaccharide chain or with a second polysaccharide chain that is already crosslinked to the first polysaccharide chain, since they are already in close proximity to each other. This is undesirable, as intramolecular crosslinking decreases the swelling ratio without a significant increase in tensile modulus.

In contrast, if long hydrophilic crosslinkers are used, when one end of the crosslinker reacts with a polysaccharide chain, the other end can move around and react with a polysaccharide chain which is significantly further away from the first polysaccharide chain, due to the flexible nature of the long crosslinker. Therefore, when a long hydrophilic crosslinker is used, it is highly likely that the second polysaccharide chain will be a different chain which has not been crosslinked to the first polysaccharide chain. This overcomes the limitations of low mobility observed when using a short crosslinker. Further, since it is unlikely for crosslinking to occur within the same polysaccharide chain or to occur multiple times between two polysaccharide chains, the amount of crosslinkers used can be reduced, while still retaining a strong polymeric network structure.

The first optionally substituted aliphatic molecule may be saturated or unsaturated, linear or branched.

The first optionally substituted aliphatic molecule may comprise an optionally substituted alkyl or optionally substituted heteroalkyl. The optionally substituted alkyl may be optionally substituted with a substituent selected from the group consisting of hydroxyl, alkyloxy, carboxyl, thioalkoxy and carboxyamide. The optionally substituted heteroalkyl may be an ether or amine.

The first optionally substituted aliphatic molecule may be a hydrophilic polymer.

The first optionally substituted aliphatic molecule may be selected from the group consisting of polyether, polyacrylamide, polyethyleneimine, polyacrylate, polymethacrylate, polyvinyl pyrrolidone and polyvinyl alcohol, each further comprising at least two hydroxy groups.

The first optionally substituted aliphatic moiety or Z may have the following structure

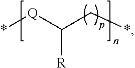

wherein
Q is —CH₂—, —O— or —NH₂—,
R is hydrogen, —OH, optionally substituted C₁ to C₆ alkyl, —C(O)OM, —C(O)NR²R³, or optionally substituted heterocycloalkyl,
R² and R³ are independently hydrogen or optionally substituted C₁ to C₆ alkyl,
M is R², Na or K.
p in an integer in the range of 1 to 6,
n is an integer in the range of 2 to 2000, and
* indicates where the moiety attaches to the rest of the spacer crosslinker.

R may be hydrogen, methyl, ethyl, propyl, butyl, pentyl or hexyl. R may be hydrogen or methyl.

R may be —C(O)OH, —C(O)ONa or —C(O)OK.

The heteroatom of the optionally substituted heterocycloalkyl may be N.

The optionally substituted heterocycloalkyl may comprise a heteroatom N and may be bonded to the rest of the optionally substituted aliphatic moiety via the N atom.

R may be selected from the group consisting of 2-pyrrolidone, 3-pyrrolidone, pyrrolidine, imidazolidine, pyrazolidine, piperidine, morpholine and diazine.

R may be C(O)NR²R³, and when R is C(O)NR²R³, R² and R³ may both be hydrogen.

p may be an integer of 1, 2, 3, 4, 5 or 6.

n may be an integer in the range of 2 to 5, 2 to 10, 2 to 20, 2 to 50, 20 to 100, 2 to 200, 2 to 500, 2 to 1000, 2 to 2000, 5 to 10, 5 to 20, 5 to 50, 5 to 100, 5 to 200, 5 to 500, 5 to 1000, 5 to 2000, 10 to 20, 10 to 50, 10 to 100, 10 to 200, 10 to 500, 10 to 1000, 10 to 2000, 20 to 50, 20 to 100, 20 to 200, 20 to 500, 20 to 1000, 20 to 2000, 50 to 100, 50 to 200, 50 to 500, 50 to 1000, 50 to 2000, 100 to 200, 100 to 500, 100 to 1000, 100 to 2000, 200 to 500, 200 to 1000, 200 to 2000, 500 to 1000, 500 to 2000 or 1000 to 2000.

The first optionally substituted aliphatic moiety or Z may have the following structure

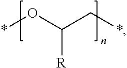

wherein
R is hydrogen or an optionally substituted C₁ to C₆ alkyl,
   n is an integer in the range of 2 to 2000, and
   * indicates where the moiety attaches to the rest of the spacer crosslinker.

The first optionally substituted aliphatic molecule may be polyethylene glycol or polypropylene glycol each further comprising at least two hydroxy groups.

Polyethylene glycol (PEG) is a polyether that is amphiphilic and soluble in water as well in many organic solvents. PEG is readily available in a wide range of molecular weights and it has been found to be nontoxic and is approved by the US Food and Drug Administration (FDA). Modified PEG having a low polydispersity index and reactive groups at both ends can be used as a long hydrophilic crosslinker to prepare hydrogels with different physical properties depending on the PEG chain length used.

The second moiety or A comprising at least two carboxylic acid groups may be derived from a second molecule having at least three carboxylic acid groups. In this context, “derived” means that the second moiety comprising at least two carboxylic acid groups is formed when one of the carboxylic acid groups of the second molecule having at least three carboxylic acid groups is reacted to form part of the linker L in formula (I).

The second molecule having at least three carboxylic acid groups may be selected from the group consisting of citric acid, pyromellitic acid, butanetetracarboxylic acid, and benzoquinonetetracarboxylic acid.

The second moiety or A comprising at least two carboxylic acid groups may be selected from the group consisting of:

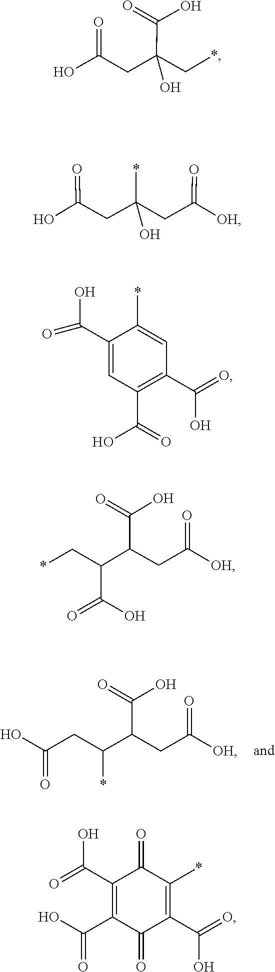

wherein * indicates where the moiety attaches to the rest of the spacer crosslinker.

L may be independently selected from the group consisting of an amide, ester, acid anhydride and thioester.

Advantageously, PEG is readily available in a wide range of molecular weights and has been found to be nontoxic and approved by the US Food and Drug Administration (FDA). Modified PEG having CA reactive groups at both ends may be used as a long hydrophilic crosslinker to prepare an SAP with different physical properties depending on the PEG chain length.

The crosslinked hydrophilic polymer may be in the form of a powder having a particle size in the range of about 0.01 mm to about 5 mm, about 0.01 mm to about 0.02 mm, about 0.01 mm to about 0.05 mm, about 0.01 mm to about 0.1 mm, about 0.01 mm to about 0.2 mm, about 0.01 mm to about 0.5 mm, about 0.01 mm to about 1 mm, about 0.01 mm to about 2 mm, about 0.02 mm to about 0.05 mm, about 0.02 mm to about 0.1 mm, about 0.02 mm to about 0.2 mm, about 0.02 mm to about 0.5 mm, about 0.02 mm to about 1 mm, about 0.02 mm to about 2 mm, about 0.02 mm to about 5 mm, about 0.05 mm to about 0.1 mm, about 0.1 mm to about 0.2 mm, about 0.1 mm to about 0.5 mm, about 0.1 mm to about 1 mm, about 0.1 mm to about 2 mm, about 0.1 mm to about 5 mm, about 0.2 mm to about 0.5 mm, about 0.2 mm to about 1 mm, about 0.2 mm to about 2 mm, about 0.2 mm to about 5 mm, about 0.5 mm to about 1 mm, about 0.5 mm to about 2 mm, about 0.5 mm to about 5 mm, about 1 mm to about 2 mm, about 1 mm to about 5 mm or about 2 mm to about 5 mm.

The crosslinked hydrophilic polymer as defined above may be biodegradable and/or biocompatible. The crosslinked hydrophilic polymer as defined above may comprise carboxymethylcellulose as the polysaccharide, a first optionally substituted aliphatic moiety derived from polyethylene glycol terminated at each end with a hydroxyl group and a second moiety derived from citric acid. Each of carboxymethylcellulose, polyethylene glycol and citric acid may be independently biodegradable and/or biocompatible and as such, the resulting crosslinked hydrophilic polymer may also be biodegradable.

The crosslinked hydrophilic polymer may be biocompatible and may be safely ingested by an animal or a human. The crosslinked hydrophilic polymer may not cause any adverse effects to the animal or human that has ingested it.

The crosslinked hydrophilic polymer, when contacted with a liquid, may form a hydrogel.

The hydrogel may have a rheological property or mechanical strength as measured by the elastic modulus G′ value in the range of about 100 Pa to about 10,000 Pa, about 100 Pa to about 500 Pa, about 100 Pa to about 1000 Pa, 100 Pa to about 2000 Pa, about 100 Pa to about 5000 Pa, about 500 Pa to about 1000 Pa, 500 Pa to about 2000 Pa, about 500 Pa to about 5000 Pa, about 500 Pa to about 10,000 Pa, about 1000 Pa to about 2000 Pa, about 1000 Pa to about 5000 Pa, about 1000 Pa to about 10,000 Pa, about 2000 Pa to about 5000 Pa, about 2000 Pa to about 10,000 Pa or about 5000 Pa to about 10,000 Pa.

The specified elastic modulus values may contribute to the feeling of fullness after ingestion of the hydrogel, similar to how coarse fibre from vegetables may confer such a sensation.

The hydrogel may have a media uptake ratio (MUR) of at least 20, at least 50, at least 70, at least 90, or at least 100. The hydrogel may have a media uptake ratio in the range of about 20 to about 200. The media may be simulated gastric fluid (SGF).

At least about 70% by mass, about 80% by mass or about 90% by mass or 100% by mass of the hydrogel may comprise the crosslinked hydrophilic polymer in the form of particles in the size range of about 0.1 mm to about 2 mm.

At least about 70% by mass, about 80% by mass or about 90% by mass or 100% by mass of the hydrogel may comprise the crosslinked hydrophilic polymer in the form of particles in the size range of about 400 μm to about 800 μm.

The hydrogel may have a tapped density in the range of about 0.2 g/mL to about 2.0 g/mL, about 0.2 g/mL to about 0.5 g/mL, about 0.2 g/mL to about 1.0 g/mL, about 0.5 g/mL to about 1.0 g/mL, about 0.5 g/mL to about 2.0 g/mL or about 1.0 g/mL to about 2.0 g/mL.

The hydrogel may have a loss on drying of about 20% (wt/wt) or less, about 10% (wt/wt) or less, about 5% (wt/wt) or less, about 2% (wt/wt) or less or about 1% (wt/wt) or less. The hydrogel may have a loss of drying in the range of about 0.1% (wt/wt) to about 20% (wt/wt).

The hydrogel may have a G′ value in the range of about 100 Pa to about 10,000 Pa and a media uptake ratio of at least 20, when determined on a sample of the crosslinked hydrophilic polymer in the form of particles whereby at least 80% by mass of the particles are in the size range of 0.1 mm to 2 mm, having a tapped density in the range of 0.5 g/mL to 1.0 g/mL and a loss on drying of 10% (wt/wt) or less.

The composition may comprise a lipase inhibitor.

A lipase inhibitor may be a substance used to reduce the activity of lipases found in the intestine. Lipases are secreted by the pancreas when fat is present. The primary role of lipase inhibitors may be to decrease the gastrointestinal absorption of fats. Fats may then tend to be excreted in faeces rather than being absorbed to be used as a source of caloric energy, and this may result in weight loss in individuals.

Lipase inhibitors may affect the amount of fat absorbed, yet may not block the absorption of a particular type of fat. Likewise, lipase inhibitors may not be absorbed into the bloodstream. Lipase inhibitors may bind to lipase enzymes in the intestine, thus preventing the hydrolysis of dietary triglycerides into monoglycerides and fatty acids. This may then reduce the absorption of dietary fat. Lipase inhibitors may covalently bond to the active serine site on lipases. This covalent bond may be strong, meaning that the lipase inhibitor may remain attached to the lipase. Lipase inhibitors may work optimally when 40% of an individual's daily caloric intake is obtained from fat. The lipase inhibitor may block absorption of 30% of total fat intake from a meal, as the lipase inhibitor bound to the lipase may pass out of the digestive tract more rapidly than fat does.

The lipase inhibitor may be selected from the group consisting of:
tetrahydrolipstatin or Orlistat [(2S,3S,5S)-5-[(S)-2-formamido4-methyl-valeryloxy]-2-hexyl-3-hydroxy-hexadecanoic 1,3 acid lactone];
lipstatin [(2S,3S,5S,7Z, 10Z)-5-[(S)-2-formamido-4-methyl-valeryloxy]-2-hexyl-3-hydroxy-7,10-hexadecadienoic 1,3 acid lactone];
FL-386 [1-(trans-4-isobutylcyclohexyl)-2-(phenylsulfonyloxy)ethenone];
WAY-121898 [4-methylpiperidine-1-carboxylic acid 4-phenoxyphenyl ester];
BAY-N-3176 [N-[3-chloro-4-(trifluoromethyl)phenyl-]N′-[3-(trifluoromethyl)-phenyl]urea];
valilactone [N-formyl-L-valine-(S)-1-[[(2S, 3S)-3-hexyl-4-oxo-2-oxetanyl]methyl]hexyl ester];
esterastin [(2S,3S,5S,7Z,10Z)-5-[(S)-2-acetamido-3-carbamoylpropionyloxy]-2-hexyl-3-hydroxy-7,10-hexadecadienoic lactone];
ebelactone A [(3S,4S)-4-[(1S,5R,7S,8R,9R,E)-8-hydroxy 1,3,5,7,9-pentamethyl-6-oxo-3-undecenyl]-3-methyl-2-oxetanone];
ebelactone B [(3S,4S)-3-ethyl-4-[(1S,5R,7S,8R,9R,E)-8-hydroxy-1,3,5,7,9-pentamethyl-6-oxo-3-undecenyl]-2-oxetanone];
RHC 80267 [1,6-di(O-(carbamoyl)cyclohexanone oxime)hexane];
cetilistat (ATL-962) [2-(hexadecyloxy)-6-methyl-4H-3,1-benzoxazin-4-one].
   and any mixture thereof.

The composition weight ratio of the crosslinked hydrophilic polymer to the lipase inhibitor may be greater than about 10, greater than about 20, greater than about 50, greater than about 100, greater than about 200, greater than about 500 or greater than about 1000. The composition weight ratio of the crosslinked hydrophilic polymer to the lipase inhibitor may be in the range of about 10 to about 20, about 10 to about 50, about 10 to about 100, about 10 to about 200, about 10 to about 500, about 10 to about 1000, about 20 to about 50, about 20 to about 100, about 20 to about 200, about 20 to about 500, about 20 to about 1000, about 50 to about 100, about 50 to about 200, about 50 to about 500, about 50 to about 1000, about 100 to about 200, about 100 to about 500, about 100 to about 1000, about 200 to about 500, about 200 to about 1000 or about 500 to about 1000.

The composition may further comprise an amylase inhibitor, a glucosidase inhibitor or any mixture thereof.

An amylase inhibitor and/or a glucosidase inhibitor may be an intestinal enzyme that slows the absorption of carbohydrates through the inhibition of enzymes responsible for their digestion. Amylase and/or glucosidase may release glucose from larger carbohydrates by hydrolysis. Amylase may hydrolyse complex starches to oligosaccharides, whereas glucosidase may hydrolyse oligosaccharides, trisaccharides and disaccharides to glucose and other monosaccharides in the small intestine. Inhibition of these enzymes may reduce the rate of digestion of complex carbohydrates. Less glucose may be absorbed because the carbohydrates are not broken down into glucose molecules. The short-term effect of using an amylase inhibitor and/or a glucosidase inhibitor may be to decrease blood glucose levels, and the long-term effects may be a reduction in HbA_{1c} levels.

The amylase inhibitor may be a glucosidase inhibitor.

The amylase inhibitor and/or the glucosidase inhibitor may be selected from the group consisting of:
acarbose [4,6-dideoxy-4-[[[1S-(1α,4α,5β,6α)]-4,5,6-trihydroxy-3-(hydroxymethyl)-2-cyclohexen-1-yl]amino]-α-D-glucopyranosyl-(1.fwdarw.4)O-α-D-glucopyranosyl-(1→4)-D-glucose];
voglibose [2(S),3(R),4(S),5(S)-tetrahydroxy-N-[2-hydroxy-1-(hydroxymethyl)-ethyl]-5-(hydroxymethyl)-1(S)-cyclohexamine];
miglitol [1,5-dideoxy-1,5-[(2-hydroxyethyl)imino]-D-glucitol];
emiglitate [1,5-dideoxy-1,5-[2-(4-ethoxycarbonylphenoxy)ethylimino]-D-glucitol];
MDL-25637 [2,6-dideoxy-2,6-imino-7-(β-D-glucopyranosyl)-D-glycero-L-guloheptitol];
camiglibose [1,5-dideoxy-1,5-(6-deoxy-1-O-methyl-α-D-glucopyranos-6-ylimino)-D-glucitol];
pradimicin Q [1,5,9,11,14-pentahydroxy-3-methyl-8,13-dioxo-5,6,8,13-tetrahydrobenzo[a]naphthacene-2-carboxylic acid];
salbostatin [1,2-dideoxy-2-[2(S),3(S),4(R)-trihydroxy-5-(hydroxymethyl)-5-cyclohexen-1(S)-ylamino]-L-glucopyranose];
   and any mixture thereof.

The composition weight ratio of the crosslinked hydrophilic polymer to the amylase inhibitor and/or the glucosidase inhibitor may be greater than about 10, greater than about 20, greater than about 50, greater than about 100, greater than about 200, greater than about 500 or greater than about 1000. The composition weight ratio of the crosslinked hydrophilic polymer to the amylase inhibitor and/or the glucosidase inhibitor may be in the range of about 10 to about 20, about 10 to about 50, about 10 to about 100, about 10 to about 200, about 10 to about 500, about 10 to about 1000, about 20 to about 50, about 20 to about 100, about 20 to about 200, about 20 to about 500, about 20 to about 1000, about 50 to about 100, about 50 to about 200, about 50 to about 500, about 50 to about 1000, about 100 to about 200, about 100 to about 500, about 100 to about 1000, about 200 to about 500, about 200 to about 1000 or about 500 to about 1000.

The composition may further comprise a pharmaceutically acceptable excipient.

The language “pharmaceutically acceptable excipient” is intended to include, but is not limited to, solvents, dispersion media, coatings, anti-bacterial and anti-fungal agents, isotonic and absorption delaying agents, diluents, fillers, thickeners, disintegrants, emulsifiers, lubricants, glidants, binders, binding agents, colouring agents, film-forming agents, preservatives, stabilizers, wetting agents, salts (to change the osmotic pressure or to act as buffers), plasticizers, anti-adherents, opacifiers and the like, and mixtures thereof. The use of such media and agents for pharmaceutically active substances is well known in the art. Except insofar as any conventional media or agent is incompatible with the composition, use thereof in the therapeutic compositions and methods of treatment and prophylaxis is contemplated. Supplementary active compounds may also be incorporated.

The excipient can be selected from, but is not limited to, agents such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue may be incorporated into sustained-release preparations and formulations.

In one example, the excipient may be an orally administrable excipient.

Fillers or diluents may include but are not limited to starches, lactose, mannitol, cellulose derivatives, microcrystalline cellulose, dextran, confectioner's sugar and the like. Different grades of lactose may include but are not limited to lactose monohydrate, lactose DT (direct tableting), lactose anhydrous, Flowlac™ (available from Meggle Products), Pharmatose™ (available from DMV) and others. Different grades of starches may include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that may be used include crystalline celluloses and powdered celluloses. Examples of crystalline cellulose products may include but are not limited to CEOLUST™ KG801, Avicel™ PH101, PH102, PH301, PH302 and PH-F20, PH-1 12 microcrystalline cellulose PH1 14, and microcrystalline cellulose PH1 12. Other useful diluents may include but are not limited to croscarmellose, sugar alcohols such as mannitol, sorbitol and xylitol, calcium carbonate, magnesium carbonate, dibasic calcium phosphate, and tribasic calcium phosphate.

Binders may include but are not limited to hydroxypropylcelluloses (Klucel™-LF), hydroxypropylcelluloses (Klucel EXF) hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

Disintegrants may include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™, FMC-Asahi Chemical Industry Co., Ltd.), crospovidones, examples of commercially available crospovidone products may include but are not limited to crosslinked povidones, Kollidon™ CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropylcelluloses may include but are not limited to low-substituted hydroxy propylcellulose LH1 1, LH21, LH31, LH22, LH32, LH20, LH30, LH32 and LH33 (all manufactured by Shin-Etsu Chemical Co., Ltd.). Other useful disintegrants may include sodium starch glycolate Type A, colloidal silicon dioxide 200, and starches.

Coloring agents may be used to color code the formulation, for example, to indicate the type and dosage of the therapeutic agent therein. Suitable coloring agents may include, without limitation, natural and/or artificial materials such as FD&C coloring agents, natural juice concentrates, pigments such as titanium oxide, silicon dioxide, iron oxides, and zinc oxide, combinations thereof, and the like.

Lubricants may include sodium stearyl fumerate, magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and combinations thereof.

One or more glidant materials, which may improve the flow of a powder blend and minimize the dosage form weight variation, may be used. Other useful glidants may include but are not limited to silicone dioxide, talc, and combinations thereof.

The final formulation, if in the solid form, may be coated or uncoated. For coating, additional excipients such as film-forming polymers, wetting agents/emulsifiers, plasticizers, anti-adherents and opacifiers may be used.

Film-forming agents may include but are not limited to cellulose derivatives such as soluble alkyl- or hydroalkylcellulose derivatives including methyl celluloses, hydroxymethyl celluloses, hydroxyethyl celluloses, hydroxy propyl celluloses, hydroxymethyethyl celluloses, hydroxypropyl methyl celluloses, sodium carboxymethyl celluloses, etc., acidic cellulose derivatives such as cellulose acetate phthalates, cellulose acetate trimellitates and methylhydroxy propylcellulose phthalates, polyvinyl acetate phthalates, etc., insoluble cellulose derivatives such as ethyl celluloses and the like, dextrins, starches and starch derivatives, polymers based on carbohydrates and derivatives thereof, natural gums such as gum Arabic, xanthans, alginates, polyacrylic acid, polyvinyl alcohol, polyvinyl acetate, polyvinylpyrrolidones, polymethacrylates and derivatives thereof (Eudragit™), chitosan and derivatives thereof, shellac and derivatives thereof, waxes and fat substances.

Wetting agents/emulsifiers may include anionic surfactants such as chenodeoxycholic acid, 1-octanesulfonic acid sodium salt, sodium deoxycholate, glycodeoxycholic acid sodium salt. N-lauroylsarcosine sodium salt, lithium dodecyl sulfate, sodium cholate hydrate, sodium dodecyl sulfate (SLS or SDS), cationic surfactants such as cetylpyridinium chloride monohydrate and hexadecyl trimethylammonium bromide, nonionic surfactants such as N-decanoyl-N-methylglucamine, octyl a-D-glucopyranoside, n-Dodecyl b-D-maltoside (DDM), polyoxyethylene sorbitan esters like polysorbates and the like. A class of nonionic surfactants particularly suitable for the subject of the invention may be made of poloxamers which are “block copolymers” of ethylene oxide and of propylene oxide units. The poloxamers which are of particular interest may have a molecular weight of between 5000 and 15500. The poloxamers may be sold in particular under the trade name Pluronic®, among which is Pluronic® F68, or poloxamer 188, which denotes a poloxamer in solid form at ambient temperature. A sorbitan ester, in particular polyoxyethylenated sorbitan ester sold under the trade name Tween®, like Tween® 20 (polyoxyethylene (20) sorbitan monolaurate or polysorbate 20) or Tween® 80 (polyoxyethylene (20) sorbitan monooleate or polysorbate 80) may also be used.

Plasticizers may include acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like.

The pharmaceutically acceptable excipient may be selected from the group consisting of filler or diluent, disintegrant, coloring agent, lubricant, binder, thickener, film-forming agent, wetting agent or emulsifier, and any mixture thereof.

The pharmaceutically acceptable excipient may be selected from the group consisting of microcrystalline cellulose, dextran, carboxymethylstarch sodium, silicon dioxide, colloidal silicon dioxide 200, talc, polyvinylpyrrolidone, polyvinylpyrrolidone K30, poloxamer such as Pluronic® F68, sodium stearyl fumerate and any mixture thereof.

The pharmaceutically acceptable excipient may be a wetting agent or an emulsifier. The wetting agent and/or emulsifier may be required to dissolve the lipase inhibitor when mixing the lipase inhibitor with the crosslinked hydrophilic polymer crosslinked with a crosslinker.

A weight ratio of the excipient to the lipase inhibitor may be in the range of about 0.01 to about 2, about 0.01 to about 0.02, about 0.01 to about 0.05, about 0.01 to about 0.1, about 0.01 to about 0.2, about 0.01 to about 0.5, about 0.01 to about 1, about 0.02 to about 0.05, about 0.02 to about 0.1, about 0.02 to about 0.2, about 0.02 to about 0.5, about 0.02 to about 1, about 0.02 to about 2, about 0.05 to about 0.1, about 0.05 to about 0.2, about 0.05 to about 0.5, about 0.05 to about 1, about 0.05 to about 2, about 0.1 to about 0.2, about 0.1 to about 0.5, about 0.1 to about 1, about 0.1 to about 2, about 0.2 to about 0.5, about 0.2 to about 1, about 0.2 to about 2, about 0.5 to about 1, about 0.5 to about 2 or about 1 to about 2.

There is also provided a method of forming the composition as defined above, comprising the step of contacting a lipase inhibitor with a hydrophilic polymer crosslinked with a crosslinker; wherein the crosslinked hydrophilic polymer has a media uptake ratio (MUR) of at least 20, and an elastic modulus (G′) value in the range of 100 Pa to 10,000 Pa, and wherein a weight ratio of the crosslinked hydrophilic polymer to the lipase inhibitor is greater than 10.

The contacting step may be mixing or spraying.

The mixing step may comprise a step of physically blending the lipase inhibitor with a filler or diluent and/or a disintegrant to form a first part. The mixing step may further comprise a step of dissolving a binder or film-forming agent and wetting agent or emulsifier in water to form a second part.

The mixing step may further comprise the step of adding the second part to the first part to form a mixture.

The mixture may be further kneaded, granulated and/or extruded to form a pellet.

The mixing step may further comprise the step of drying and sieving the pellet.

The mixing step may further comprise the step of mixing the pellet with the crosslinked hydrophilic polymer.

The spraying step may comprise spray coating the crosslinked hydrophilic polymer in particle form with a binder solution comprising the lipase inhibitor, a binder or film-forming agent and wetting agent or emulsifier, to form a coated particle.

The spraying step may further comprise the step of mixing the coated particle with a lubricant to form a coated particle mixture.

The spraying step may further comprise the step of drying and sieving the coated particle mixture.

There is also provided a composition obtained by the method as defined above.

There is also provided a capsule comprising the composition as defined above.

Each dosage unit may comprise the composition as defined above at an amount in the range of about 400 mg to about 5500 mg, about 400 mg to about 750 mg, about 400 mg to about 1000 mg, about 400 mg to about 1250 mg, about 400 mg to about 1500 mg, about 400 mg to about 1750 mg, about 400 mg to about 2000 mg, about 400 mg to about 3000 mg, about 750 mg to about 1000 mg, about 750 mg to about 1250 mg, about 750 mg to about 1500 mg, about 750 mg to about 1750 mg, about 750 mg to about 2000 mg, about 750 mg to about 3000 mg, about 750 mg to about 5500 mg, about 1000 mg to about 1250 mg, about 1000 mg to about 1500 mg, about 1000 mg to about 1750 mg, about 1000 mg to about 2000 mg, about 1000 mg to about 3000 mg, about 1000 mg to about 5500 mg, about 2000 mg to about 3000 mg, about 2000 mg to about 5500 mg or about 3000 mg to about 5500 mg.

The capsule may be made of gelatin and may be used for oral administration of the composition to a subject.

There is also provided a method of treating obesity, pre-diabetes, diabetes, non-alcoholic fatty liver disease (NAFLD), non-alcoholic steatohepatitis (NASH), or chronic idiopathic constipation, or of reducing caloric intake or improving glycemic control in a subject in need thereof, comprising the step of administering to the subject a therapeutically effective amount of the composition as defined above.

As used herein the term “treatment”, refers to any and all uses which remedy a disease state or symptoms, prevent the establishment of disease, or otherwise prevent, hinder, retard, or reverse the progression of disease or other undesirable symptoms in any way whatsoever.

One skilled in the art would be able to determine effective, non-toxic dosage levels of the composition and an administration pattern which would be suitable for treating the diseases or conditions to which the composition is applicable.

Further, it will be apparent to one of ordinary skill in the art that the optimal course of treatment, such as the number of doses of the composition given per day for a defined number of days, can be ascertained using convention course of treatment determination tests.

The composition may be administered alone. Alternatively, the composition may be administered as a pharmaceutical, veterinarial, or industrial formulation. The composition may also be present as suitable salts, including pharmaceutically acceptable salts.

In one example, the composition is to be administered orally. The composition can be administered orally, for example, with an inert diluent or an assimilable edible carrier. The composition and other ingredients can also be enclosed in a hard or soft shell gelatin capsule, compressed into tablets, or incorporated directly into an individual's diet. For oral therapeutic administration, the composition can be incorporated with excipients and used in the form of ingestible tablets, buccal tablets, troches, capsules, elixirs, suspensions, syrups, wafers, and the like.

It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of the composition is calculated to produce the desired therapeutic effect in association with the required pharmaceutical excipient. The composition may be formulated for convenient and effective administration in effective amounts with a suitable pharmaceutically acceptable excipient in an acceptable dosage unit. In the case of compositions containing supplementary active ingredients, the dosages are determined by reference to the usual dose and manner of administration of the said ingredients.

The dosage unit form may be a solid form, for example, as pellets, tablets, capsules, lozenges, wafers or crackers or in liquid form, for example, as solutions, or emulsions.

Depending on the degree of swelling, the amount of crosslinked hydrophilic polymer in each dosage unit form to be administered to achieve the desired effect may be in the range of about 400 mg to about 55,000 mg, about 400 mg to about 1000 mg, about 400 mg to about 1500 mg, about 400 mg to about 2000 mg, about 400 mg to about 2500 mg, about 400 mg to about 3000 mg, about 400 mg to about 5000 mg, about 400 mg to about 10,000 mg, about 400 mg to about 25,000 mg, about 1000 mg to about 1500 mg, about 1000 mg to about 2000 mg, about 1000 mg to about 2500 mg, about 1000 mg to about 3000 mg, about 1000 mg to about 5000 mg, about 1000 mg to about 10,000 mg, about 1000 mg to about 25,000 mg, about 1000 mg to about 50,000 mg, about 1500 mg to about 2000 mg, about 1500 mg to about 2500 mg, about 1500 mg to about 3000 mg, about 1500 mg to about 5000 mg, about 1500 mg to about 10,000 mg, about 1500 mg to about 25,000 mg, about 1500 mg to about 55,000 mg, about 2000 mg to about 2500 mg, about 2000 mg to about 3000 mg, about 2000 mg to about 5000 mg, about 2000 mg to about 10,000 mg, about 2000 mg to about 25,000 mg, about 2000 mg to about 55,000 mg, about 2500 mg to about 3000 mg, about 2500 mg to about 5000 mg, about 2500 mg to about 10,000 mg, about 2500 mg to about 25,000 mg, about 2500 mg to about 55,000 mg, about 3000 mg to about 5000 mg, about 3000 mg to about 10,000 mg, about 3000 mg to about 25,000 mg, about 3000 mg to about 55,000 mg, about 5000 mg to about 10,000 mg, about 5000 mg to about 25,000 mg, about 5000 mg to about 55,000 mg, about 10,000 mg to about 25,000 mg, about 10,000 mg to about 55,000 mg or about 25,000 mg to about 55,000 mg.

The amount of lipase inhibitor in each dosage unit form to be administered to achieve the desired effect may be in the range of about 10 mg to about 1000 mg, about 10 mg to about 20 mg, about 10 mg to about 50 mg, about 10 mg to about 100 mg, about 10 mg to about 200 mg, about 10 mg to about 500 mg, about 20 mg to about 50 mg, about 20 mg to about 100 mg, about 20 mg to about 200 mg, about 20 mg to about 500 mg, about 20 mg to about 1000 mg, about 50 mg to about 100 mg, about 50 mg to about 200 mg, about 50 mg to about 500 mg, about 50 mg to about 1000 mg, about 100 mg to about 200 mg, about 100 mg to about 500 mg, about 100 mg to about 1000 mg, about 200 mg to about 500 mg, about 200 mg to about 1000 mg or about 500 mg to about 1000 mg.

The amount of amylase inhibitor and/or glucosidase inhibitor in each dosage unit form to be administered to achieve the desired effect may be in the range of about 10 mg to about 1000 mg, about 10 mg to about 20 mg, about 10 mg to about 50 mg, about 10 mg to about 100 mg, about 10 mg to about 200 mg, about 10 mg to about 500 mg, about 20 mg to about 50 mg, about 20 mg to about 100 mg, about 20 mg to about 200 mg, about 20 mg to about 500 mg, about 20 mg to about 1000 mg, about 50 mg to about 100 mg, about 50 mg to about 200 mg, about 50 mg to about 500 mg, about 50 mg to about 1000 mg, about 100 mg to about 200 mg, about 100 mg to about 500 mg, about 100 mg to about 1000 mg, about 200 mg to about 500 mg, about 200 mg to about 1000 mg or about 500 mg to about 1000 mg.

The amount of pharmaceutical excipient in each dosage unit form to be administered to achieve the desired effect may be in the range of about 20 mg to about 2000 mg, about 20 mg to about 50 mg, about 20 mg to about 100 mg, about 20 mg to about 200 mg, about 20 mg to about 500 mg, about 20 mg to about 1000 mg, about 50 mg to about 100 mg, about 50 mg to about 200 mg, about 50 mg to about 500 mg, about 50 mg to about 1000 mg, about 50 mg to about 2000 mg, about 100 mg to about 200 mg, about 100 mg to about 500 mg, about 100 mg to about 1000 mg, about 100 mg to about 2000 mg, about 200 mg to about 500 mg, about 200 mg to about 1000 mg, about 500 mg to about 2000 mg or about 1000 mg to about 2000 mg.

The dosage unit form, in solid form, may be further coated with a pharmaceutically acceptable excipient. The coating of a dosage unit form may be carried out in a fluid bed processor using a bottom spray, a top spray, or a tangential spray attachment. The flowability, processability and other characteristics of the dosage unit form may be readily controlled through the choice of appropriate pharmaceutically acceptable excipients onto which the dosage unit forms are coated; and by varying the process variables such as the spray rate and the degree of fluidization.

In one example, the composition is to be administered in single or multiple doses. In one example, the composition is to be administered in a single, double, triple or quadruple doses. In another example, the composition can be, or is to be, administered at an interval of, but not limited to, hourly, daily, twice daily, thrice daily, 4 times a day, every second day, every third day, every fourth day, every fifth day, every sixth day, weekly, biweekly, bimonthly, monthly, or combinations thereof.

Generally, an effective dosage per 24 hours may be in the range of about 0.001 mg to about 500 mg per kg body weight; about 0.001 mg to about 0.01 mg per kg body weight, about 0.001 mg to about 0.1 mg per kg body weight, about 0.001 mg to about 1 mg per kg body weight, about 0.001 mg to about 10 mg per kg body weight, about 0.001 mg to about 100 mg per kg body weight, about 0.01 mg to about 500 mg per kg body weight; about 0.01 mg to about 0.1 mg per kg body weight, about 0.01 mg to about 1 mg per kg body weight, about 0.01 mg to about 10 mg per kg body weight, about 0.01 mg to about 100 mg per kg body weight, about 0.1 mg to about 500 mg per kg body weight; about 0.1 mg to about 1 mg per kg body weight, about 0.1 mg to about 10 mg per kg body weight, about 0.1 mg to about 100 mg per kg body weight, about 1 mg to about 500 mg per kg body weight; about 1 mg to about 10 mg per kg body weight, about 1 mg to about 100 mg per kg body weight, about 10 mg to about 500 mg per kg body weight; about 10 mg to about 100 mg per kg body. More suitably, an effective dosage per 24 hours may be in the range of about 10 mg to about 500 mg per kg body weight; about 10 mg to about 250 mg per kg body weight; about 50 mg to about 500 mg per kg body weight; about 50 mg to about 200 mg per kg body weight; or about 50 mg to about 100 mg per kg body weight.

An effective dosage routine may be once a week, twice a week, thrice a week, daily, twice daily or thrice daily.

An effective dosage routine may be twice or thrice daily, and each dose may comprise one, two, three, four or five dosage unit forms as defined above.

Each dose may comprise about 1 g to about 6 g, about 1 g to about 2 g, about 1 g to about 3 g, about 1 g to about 4 g, about 1 g to about 5 g, about 2 g to about 3 g, about 2 g to about 3 g, about 2 g to about 4 g, about 2 g to about 5 g, about 2 g to about 5 g, about 2 g to about 6 g, about 3 g to about 4 g, about 3 g to about 5 g, about 3 g to about 6 g, about 4 g to about 5 g, about 4 g to about 6 g, or about 5 g to about 6 g of the composition as defined above.

Each dose may comprise 2 to 8 dosage unit forms, 2 to 3 dosage unit forms, 2 to 4 dosage unit forms, 2 to 5 dosage unit forms, 2 to 6 dosage unit forms, 2 to 7 dosage unit forms, 3 to 4 dosage unit forms, 3 to 5 dosage unit forms, 3 to 6 dosage unit forms, 3 to 7 dosage unit forms, 3 to 8 dosage unit forms, 4 to 5 dosage unit forms, 4 to 6 dosage unit forms, 4 to 7 dosage unit forms, 4 to 8 dosage unit forms, 5 to 6 dosage unit forms, 5 to 7 dosage unit forms, 5 to 8 dosage unit forms, 6 to 7 dosage unit forms, 6 to 8 dosage unit forms or 7 to 8 dosage unit forms comprising the composition as defined above.

Each dose may comprise about 2.25 g of the composition as defined above, administered as 4 dosage unit forms, wherein each dosage unit form in the form of a capsule may comprise about 0.5625 g of the composition as defined above.

Each dose may comprise about 2.24 g of the composition as defined above, administered as 4 dosage unit forms, wherein each dosage unit form in the form of a capsule may comprise about 0.56 g of the composition as defined above.

Each dose may comprise about 2.16 g of the composition as defined above, administered as 4 dosage unit forms, wherein each dosage unit from in the form of a capsule may comprise about 0.54 g of the composition as defined above.

The composition may be administered before a meal. The composition may be administered about 10 minutes to about 1 hour, about 10 minutes to about 20 minutes, about 10 minutes to about 30 minutes, about 10 minutes to about 45 minutes, about 20 minutes to about 30 minutes, about 20 minutes to about 45 minutes, about 20 minutes to about 1 hour, about 30 minutes to about 45 minutes, about 30 minutes to about 1 hour or about 45 minutes to about 1 hour before a meal.

The composition may be administered with water. The composition may be administered with about 100 mL to about 700 mL, about 100 mL to about 250 mL, about 100 mL to about 500 mL, about 250 mL to about 500 mL, about 250 mL to about 700 mL or about 500 mL to about 700 mL of water.

The composition of the invention can be used in combination with other known treatments for the disease or condition. Combinations of active agents, including the composition, can be synergistic.

The subject can be, but is not limited to, an animal that is as risk or is suffering from obesity, pre-diabetes, diabetes, non-alcoholic fatty liver disease (NAFLD), non-alcoholic steatohepatitis (NASH), or chronic idiopathic constipation. The subject may further be in the need to reduce caloric intake or improve glycemic control. In one example, the animal is a human.

There is also provided the composition as defined above for use in the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

There is also provided the use of the composition as defined above in the manufacture of a medicament for the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

There is also provided a method of weight-loss or a method of improving the body appearance in a healthy subject, comprising the step of orally administering to the subject the composition as defined above.

### EXAMPLES

Non-limiting examples of the invention will be further described in greater detail by reference to specific Examples, which should not be construed as in any way limiting the scope of the invention.

### Materials

Carboxymethylcellulose (CMC) sodium salt was obtained from Ashland Inc. Polyethylene glycol (PEG) was purchased from Sigma-Aldrich and used without further modification. Citric acid (CA) was obtained from Tokyo Chemical Industry (TCI) and used without further modification. Control Orlistat capsules Xenical (120 mg) were purchased from Roche (Lot:M2383M3). Orlistat as active pharmaceutical ingredient (API) was purchased from Zhejiang Haizheng Pharmaceutical Co., and excipients including microcrystalline cellulose (MCC), polyvinylpyrrolidone (PVP K30), carboxymethylstarch sodium (CMS-Na) and sodium dodecyl sulfate (SDS) were obtained from Ashland Inc. Cross-linked polyacrylate SAP (Waste Lock® 770) was purchased from M2 Polymer Technologies. Other drug fabrication and testing associated chemicals were purchased from Sigma-Aldrich and used as received.

### Example 1: Synthesis

### Synthesis of Crosslinker

Citric acid (CA, 1 g) was dissolved into 10 mL DI water, then polyethylene glycol (PEG, 10 g) was weighed and mixed with the CA solution. The fully dissolved solution was charged in a flask of a rotary evaporator (IKA) with a silicone oil bath. The solution in the rotary flask was heated at 100° C. for 0.5 hours, then the oil bath temperature was increased to 120° C. gradually. Without condensation, all the water in the flask evaporated after 2 hours, resulting in a viscous yellow coloured paste in the flask. Once cooled down to room temperature (RT), the resultant viscous paste was dissolved in deionised (DI) water to form a 50 mL solution, from which 1.0 mL (equivalent to 20 mg CA) was used for further crosslinking reaction.

### Preparation of Super Absorbent Polymer (SAP)

Deionised (DI) water (700 mL) was added to a 1 L beaker and stirred with an ANGNI electric mixer at 60 rpm. Solutions of the spacer crosslinkers with equivalent citric acid content (equivalent to 20 mg CA) was added to the water. CMC (10 g) was then added to the solution and the resulting mixture was agitated at room temperature at 120 rpm for 2 hours and then at 60 rpm for 24 hours. The final homogenised solution was poured into a stainless steel tray with a solution thickness of less than 2 cm. The tray was placed in a convection oven (Lantian) at 50° C. for 24 hours. The tray was removed from the oven, and the dried CMC sheet was inverted and the tray was placed back in the oven and maintained at 50° C. for 12 to 24 hours until no change in weight was observed.

After full desiccation, the CMC sheet was ground by means of a cutting blender (Philips). The granulated material was sieved to a particle size of from 0.1 mm to 2 mm, then spread on the tray and crosslinked in the convection oven (Binder) at 120° C. for 2 to 4 hours. The crosslinked polymer hydrogel thus obtained was washed with DI water over 4 hours by changing the washing solution 2 times to remove unreacted reagents. The washing stage increased the hydrogel's media uptake capacity by increasing the relaxation of the network. After washing, the hydrogel was placed on a tray and placed in an oven (Lantian) at 50° C. for 12 to 24 hours until no change in weight was observed. The dried hydrogel aggregates were ground and sieved to a particle size from 0.1 mm to 1 mm.

### Preparation of Composition and Capsule: Method 1

First step for this method was to prepare drug pellets according to Examples 2 to 4, 6 and 7 below. Taking Formulation A of Example 2 as a representative example, Orlistat (API, 120 g), microcrystalline cellulose (MCC, 93.6 g), and carboxymethylstarch sodium (CMS-Na, 7.2 g) were blended in a dry mixer (Xinyite, Shenzhen) for 1 hour to obtain Part A. Polyvinylpyrrolidone (PVP K30, 12 g) and sodium dodecyl sulphate (SDS, 7.2 g) were dissolved in 100 mL of purified water and homogenized to obtain Part B. Part B was then added to Part A and kneaded using a wet mixer (Xinyite, Shenzhen). The resulting paste was granulated and passed through an extruder. The extrudate was then passed through a spheronizer (Xinyite, Shenzhen) to form pellets. The obtained wet pellets were further dried in a fluidized bed dryer (Xinyite, Shenzhen) at 30° C. for 30 minutes with talc, then sieved. The 0.1-0.8 mm size fractions were collected as drug pellets.

The as-prepared crosslinked carboxymethylcellulose (X-CMC) SAP particles (2 kg) were mixed with the drug pellets (240.24 g) and homogenized thoroughly. The resulting particle mixture was infused into hard gelatin capsules (using a machine from Huaxu, Wenzhou) in the amounts indicated in Example 2 and sealed properly for further testing or evaluation.

### Preparation of Composition and Capsule: Method 2

An alternate method to the above-mentioned two-stepped process to obtain the composition and capsule, was to spray coat the SAP particles with a binder solution containing the lipase inhibitor. Briefly, Orlistat (API, 120 g), polyvinylpyrrolidone (PVP K30, 30 g) and sodium dodecyl sulphate (SDS, 8 g) were dissolved in 300 mL of purified water and homogenized to obtain a binder solution. The as-prepared SAP dry particles (400 g) were blown into the chamber of a fluidized bed (Xinyite, Shenzhen) from the bottom, while the binder solution was sprayed from the top and mixed with the SAP particles uniformly. After drying at 30° C. for 20 minutes, the drug coated SAP particles were collected from the bottom then mixed with sodium stearyl fumarate (2 g). The 0.1-0.8 mm size fractions were collected and infused into hard gelatin capsules (using a machine from Huaxu, Wenzhou) in the amounts indicated in Example 5 and sealed properly for further testing or evaluation.

### Equilibrium Swelling Testing

Media uptake measurements were performed on samples in solid form soaked for 30 minutes in aqueous media. Standard Simulated Gastric Fluid (SGF) was prepared by mixing 7 mL HCl 37%, 2 g NaCl and 3.2 g pepsin in deionised (DI) water. After solid dissolution, more water was added to achieve a volume of 1 L. Diluted SGF (Di-SGF) was prepared by mixing 8 units DI water with 1 unit of SGF, then simulating gastric fluid after water intake with pills/capsules containing the dried SAP.

Media uptake ratio (MUR) of SAP in Di-SGF was determined as follows: A dried glass funnel was placed on a support and 40 g of purified water was poured into the funnel. Once no further droplets were detected in the neck of the funnel (about 5 minutes), the funnel was placed into an empty and dry glass beaker (beaker #1), which was placed on a tared scale to record the weight of the empty apparatus (W1). 40 g of DI-SGF solution was prepared as described above and placed in beaker #2.0.25 g of SAP was accurately weighed using weighing paper. The SAP was added to beaker #2 and stirred gently for 30 minutes with a magnetic stirrer without generating vortices. The stir bar was removed from the resulting suspension, the funnel was placed on a support and the suspension was poured into the funnel to allow the material to drain for 10±1 minute. The funnel containing the drained material was placed inside beaker #1 and weighed (W2). The Media Uptake Ratio (MUR) was calculated according to: MUR=(W2−W1)/0.25. The determination was made in triplicate.

### Mechanical Strength Testing

Viscoelastic properties of the SAP hydrogels were determined according to the protocol set below. Hydrogels were freshly prepared according to MUR testing methods described above for equilibrium swelling. Briefly, 0.25 g of SAP powder was soaked with 40 g of DI-SGF solution and stirred for 30 minutes. The swelled suspension was poured into a filtration funnel and drained for 10 minutes, and the resulting hydrogel was collected for rheological tests.

Small deformation oscillation measurements were carried out with a rheometer (TA Discovery HR-30), equipped with a Peltier plate, lower and upper flat plates (Cross-hatching pattern) with 40 mm diameter. All measurements were performed with a gap of 4 mm with a Peltier sensor at 25° C. The elastic modulus, G′, was obtained over a frequency range of 0.1-50 rad/see and the strain was fixed at 0.1%. The hydrogels were subjected to a sweep frequency test with the rheometer and the value at an angular frequency of 10 rad/s was determined. The determination was made in triplicate. The reported G′ value is the average of the three determinations.

### Drug Dissolution Testing Protocol

Drug dissolution was tested according to USP40-NF35 (Orlistat capsule). Briefly, the capsule was immersed in a dissolution cup filled with liquid medium. Temperature and bladder stirring speed were set as outlined below. After a certain period, 5 mL of the solution was extracted and filtered before proceeding with HPLC quantitative testing to analyze the active pharmaceutical ingredient (API) concentration.

Specifically, the following parameters were used:
Medium: 3% sodium dodecyl sulfate and 0.5% sodium chloride in water. To each 10 L of media, 1-2 drops of n-octanol was added, and adjusted with phosphoric acid to a pH of 6.0. The dissolution medium volume was fixed at 900 mL.
Stirring speed: 75 rpm
Stirring Time: 45 minutes
Mobile phase: Acetonitrile and water (860:140)
Standard solution: About 13 mg of Orlistat reference standard (RS) was transferred to a 100 mL volumetric flask. This was dissolved in 2 mL of acetonitrile and diluted with the Medium to volume.
Sample solution: A portion of the solution being tested was passed through a suitable filter of 0.2 μm pore size.
Flow rate: 2.0 mL/min
Injection size: 50 mL
Relative standard deviation: <2.0%
The percentage of the labeled amount of Orlistat (C₂₉H₅₃NO₅) dissolved was calculated using the following equation:

$$Result = ( {r}_{U} / {r}_{S} ) \times ( {C}_{S} / L ) \times V \times 100$$

r_{U}=peak response from the Sample solution
   r_{S}=peak response from the Standard solution
   C_{S}=concentration of the Standard solution (mg/mL)
   L=label claim (mg/Capsule)
   V=volume of Medium, 900 mL
Tolerances: >75% (Q) of the labeled amount of Orlistat was dissolved.

### Example 2: Formulation A

TABLE 1
Components of Formulation A
Example A

| | |
|---|---|
| Dosage unit (pill) | 4 |
| SAP particle (mg) | |
| X-CMC (self-prepared) | 2000 |
| Drug pellet (mg) | |
| Orlistat | 120 |
| Microcrystalline cellulose | 93.6 |
| Polyvinylpyrrolidone K30 | 12 |
| Carboxymethylstarch sodium | 7.2 |
| Sodium dodecyl sulfate | 7.2 |
| Talc | 0.24 |
| Sub-total (mg) | 240.24 |
| Total (mg) | 2240.24 |

### Example 3: Formulation B

TABLE 2
Components of Formulation B
Example B

| | |
|---|---|
| Dosage unit | 4 |
| SAP particle (mg) | |
| PAA (Waste Lock ® 770) | 2000 |
| Drug pellet (mg) | |
| Orlistat | 120 |
| Dextran | 93.6 |
| Polyvinylpyrrolidone K30 | 12 |
| Carboxymethylstarch sodium | 7.2 |
| Pluronic F68 | 7.2 |
| Silicon dioxide | 0.24 |
| Sub-total (mg) | 240.24 |
| Total (mg) | 2240.24 |

### Example 4: Formulation C

TABLE 3
Components of Formulation C
Example C

| | |
|---|---|
| Dosage unit | 4 |
| SAP particle (mg) | |
| X-CMC (self-prepared) | 2000 |
| Drug pellet (mg) | |
| Orlistat | 60 |
| Acarbose | 60 |
| Microcrystalline cellulose | 93.6 |
| Polyvinylpyrrolidone K30 | 12 |
| Carboxymethylstarch sodium | 7.2 |
| Sodium dodecyl sulfate | 7.2 |
| Talc | 0.24 |
| Sub-total (mg) | 240.24 |
| Total (mg) | 2240.24 |

### Example 5: Formulation D

TABLE 4
Components of Formulation D
Example D

| | |
|---|---|
| Dosage unit | 4 |
| SAP particle (mg) | |
| X-CMC (self-prepared) | 2000 |
| Drug pellet (mg) | |
| Orlistat | 120 |
| Polyvinylpyrrolidone K30 | 30 |
| Sodium dodecyl sulfate | 8 |
| Sodium stearyl fumarate | 2 |
| Sub-total (mg) | 160 |
| Total (mg) | 2160 |

### Example 6: Formulation E

TABLE 5
Components of Formulation E
Example E

| | |
|---|---|
| Dosage unit | 4 |
| SAP particle (mg) | |
| X-CMC (self-prepared) | 2000 |
| Drug pellet (mg) | |
| Orlistat | 60 |
| Microcrystalline cellulose | 70 |
| Polyvinylpyrrolidone K30 | 12 |
| Carboxymethylstarch sodium | 4.5 |
| Sodium dodecyl sulfate | 3.5 |
| Sodium stearyl fumarate | 0.5 |
| Sub-total (mg) | 150.5 |
| Total (mg) | 2150.5 |

### Example 7: Formulation F

TABLE 6
Components of Formulation F
Example F

| | |
|---|---|
| Dosage unit | 4 |
| SAP particle (mg) | |
| X-CMC (self-prepared) | 2000 |
| Drug pellet (mg) | |
| Orlistat | 60 |
| Microcrystalline cellulose | 116.4 |
| Polyvinylpyrrolidone K30 | 10 |
| Carboxymethylstarch sodium | 10 |
| Sodium dodecyl sulfate | 3.6 |
| Sodium stearyl fumarate | 0.5 |
| Sub-total (mg) | 200.5 |
| Total (mg) | 2200.5 |

### Example 8: Evaluation of Formulation A

Compared to the control Orlistat capsule Xenical (Roche), formulation A contains about 90% super absorbent polymer (SAP) hydrogels which can disintegrate and swell very fast in contact with aqueous simulated gastric fluid (SGF). Even with about 10% drug pellets which do not contribute to the swelling ratio, formulation A still had a media uptake ratio (MUR) of about 117.8 by weight, which was close to pure SAP hydrogels. This shows that the combination of the SAP with Orlistat drug pellets did not affect the water absorption capability of the SAP in the acidic environment of the stomach. Therefore, the SAP hydrogels were shown to be able to perform as a device for occupying gastric space, together with Orlistat. A similar observation could be made with formulation B (discussed further below), which uses a polyacrylate SAP instead of crosslinked carboxymethylcellulose (X-CMC).

According to the USP40-NF35 (Orlistat capsule) testing method for drug dissolution, in vitro dissolution (DS) of the control Orlistat capsule Xenical and formulation A were both about 45 minutes and were very close (100-110%), both passing the >75% requirement for tolerance. In other words, the incorporated SAP hydrogel did not affect the Orlistat's dissolution and bioavailability characteristics.

### Example 9: Evaluation of Formulations B to F

Other embodiments of this disclosure include the usage of other non-polysaccharide SAP hydrogels and incorporation of additional active pharmaceutical ingredients (APIs) such as amylase or glucosidase inhibitors to reduce the caloric intake originating from carbohydrates. Formulations B and C described in Examples 3 and 4, respectively, provide the composition details on such embodiments, respectively. Similar drug pellets and capsulation processes as that described in Example 1 were applied.

For formulation D in Example 5, SAP hydrogels were directly coated with lipase inhibitor which optimized the manufacturing process and improved the uniformity of drug dispersion.

Formulations E and F in Examples 6 and 7, respectively, provide the composition details of 60 mg dosage Orlistat capsules with 40% and 30% API concentrations in the drug pellets, respectively. The commercially available counterpart Orlistat 60 mg capsule is from GlaxoSmithKline (GSK) and branded as Alli®, which is approved by the US Food and Drug Administration (FDA) and sold as an over the counter (OTC) product for weight loss.

As summarised in Table 7 below, it can be seen that the fundamental properties such as dissolution (DS), MUR and G′ of formulations B to F were similar to that of formulation A, which proves the versatility and flexibility of the inventive compositions.

TABLE 7
Comparison of the Orlistat dissolution (DS), media uptake ratio (MUR)
and elastic modulus (G′) characteristics of formulations A to F

| | | Drug components | | | | | |
|---|---|---|---|---|---|---|---|
| Sample | | API: | API: | | | | G′ |
| # | SAP | Orlistat | Acarbose | Excipients | DS (%) | MUR | (Pa) |
| Xenical | / | 50% | / | 50% | 99.8 | / | |
| A | 100% | 50% | / | 50% | 110.4 | 117.8 | 1822 |
| B | 100% | 50% | / | 50% | 98.8 | 134.4 | 2465 |
| C | 100% | 25% | 25% | 50% | 105.1 | 114.2 | 1829 |
| D | 100% | 75% | / | 25% | 115.3 | 110.6 | 1874 |
| E | 100% | 40% | / | 60% | 97.6 | 122.0 | 1722 |
| F | 100% | 30% | / | 70% | 109.5 | 118.8 | 1853 |

### Example 10: Effect of Excipient

An orthogonal experiment was further carried out to study the effects of the excipient on formulation A, by measuring the Orlistat dissolution, MUR and G′ results. Specifically, the amount of the excipients in formulation A were varied, as shown in Table 8 below, and it can be seen that the disintegrant (CMS-Na) and surfactant (SDS) concentrations cast a more significant impact on the dissolution characteristics of the formulation. However, all samples in Table 8 passed the dissolution requirement of 75% as required. The overall physical properties of the SAP in the samples remained stable within a narrow range of MUR between 105-130 and G′ between 1650-1850 Pa.

TABLE 8
Orlistat dissolution (DS), media uptake ratio (MUR) and elastic modulus
(G′) characteristics of formulation A with varying amounts of excipient

| Sample | SAP | Drug Pellets (60 mg) | | | | | | | |
|---|---|---|---|---|---|---|---|---|---|
| # | (500 mg) | API | MCC | CMS-Na | SDS | PVP K30 | DS (%) | MUR | G′ (Pa) |
| A | 100% | 50% | 39% | 3% | 3% | 5% | 110.4 | 117.8 | 1822 |
| A-1 | 100% | 50% | 44% | 1% | 2% | 3% | 108.6 | 122.2 | 1684 |
| A-2 | 100% | 50% | 40% | 1% | 4% | 5% | 87.7 | 109.6 | 1897 |
| A-3 | 100% | 50% | 36% | 1% | 6% | 7% | 107.5 | 107.4 | 1916 |
| A-4 | 100% | 50% | 40% | 3% | 4% | 3% | 91.4 | 118.8 | 1819 |
| A-5 | 100% | 50% | 36% | 3% | 6% | 5% | 99.9 | 114.2 | 1837 |
| A-6 | 100% | 50% | 38% | 3% | 2% | 7% | 111.9 | 125.4 | 1680 |
| A-7 | 100% | 50% | 36% | 5% | 6% | 3% | 104.8 | 116.0 | 1795 |
| A-8 | 100% | 50% | 38% | 5% | 2% | 5% | 112.0 | 126.6 | 1665 |
| A-9 | 100% | 50% | 34% | 5% | 4% | 7% | 116.4 | 123.0 | 1702 |

### Example 11: Human Volunteer Research on Pill Formulation A

To verify the synergistic effects between the SAP hydrogels and lipase inhibitors on the treatment of obesity and gastrointestinal adverse effects, two middle aged healthy male volunteers tested formulation A against the control Orlistat capsule Xenical, on a normal average mixed diet over 12 weeks. For administration of formulation A, volunteer I consumed 500 ml of water with 4 capsules (total dosage containing 2 g SAP and 120 mg Orlistat) and waited at least 30 minutes before the meal. Volunteer I took the Xenical capsule (containing 120 mg Orlistat) according to the manufacturer's instructions. As shown in Table 9, a significant amount of body weight loss was observed for volunteer I compared to volunteer II (5.6% and 3.4%, respectively) after 12 weeks, despite their similar initial body mass index (BMI). Considering the same dosage of lipase inhibitor was administrated for both volunteers, the significant increase in weight loss is attributed to the SAP hydrogels which functioned as a gastric space-occupying device and helped volunteer I reducing caloric intake.

Both quantitative (stool fat %) and qualitive (life quality score) evaluations of steatorrhea, which is a side-effect associated with ingestion of Orlistat, have been applied to the volunteers. The comparative results fully demonstrated the significant advantages of formula A against the control: stool fat % only slightly increased from 15 to 19% for volunteer I, while it almost doubled for volunteer II with the same Orlistat dosage.

TABLE 9
Comparison of the effect on human of formula A with Xenical
Human research

| Volunteer | I (Formula A) | II (Xenical) |
|---|---|---|
| Body weight change (%) | | |
| W1 | 0.0 | 0.0 |
| W2 | −0.7 | −0.4 |
| W3 | −1.3 | −0.7 |
| W4 | −2.1 | −0.9 |
| W5 | −2.6 | −1.5 |
| W6 | −3.2 | −1.9 |
| W7 | −3.6 | −2.3 |
| W8 | −4.0 | −2.5 |
| W9 | −4.3 | −2.9 |
| W10 | −4.9 | −3.1 |
| W11 | −5.3 | −3.3 |
| W12 | −5.6 | −3.4 |
| Stool Fat (%), 48 h | | |
| W1 | 15% | 12% |
| W8 | 19% | 23% |
| Life quality score (1-10) | | |
| W4 | 7.2 | 5.3 |
| W8 | 7.5 | 5.2 |
| W12 | 8.1 | 5.7 |

*W stands for week

### INDUSTRIAL APPLICABILITY

This invention may be used in the treatment of obesity, pre-diabetes, diabetes, non-alcoholic fatty liver diseases, non-alcoholic steatohepatitis, chronic idiopathic constipation, and in reducing caloric intake or improving glycemic control.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A pharmaceutical composition comprising:
a lipase inhibitor; and
a hydrophilic polymer crosslinked with a crosslinker,
wherein the crosslinked hydrophilic polymer has a media uptake ratio (MUR) of at least 20, and an elastic modulus (G′) value in the range of 100 Pa to 10,000 Pa, and
wherein a weight ratio of the crosslinked hydrophilic polymer to the lipase inhibitor is greater than 10.

2. The composition according to claim 1, wherein the crosslinked hydrophilic polymer is a hydrogel.

3. The composition according to claim 1, wherein the crosslinked hydrophilic polymer is in the form of a powder having a particle size in the range of 0.01 mm to 5 mm.

4. The composition according to claim 1, wherein the hydrophilic polymer of the crosslinked hydrophilic polymer is selected from the group consisting of polysaccharide, polyacrylate, polyacrylamide, polymer of ethylene maleic anhydride, polyvinyl alcohol, polyvinylpyrrolidone, crosslinked polyethylene oxide, starch grafted polyacrylonitrile and any copolymer thereof.

5. The composition according to claim 1, wherein the hydrophilic polymer of the crosslinked hydrophilic polymer is a polysaccharide that is or is a derivative of a compound selected from the group consisting of starch, hydroxyethyl starch, hydroxypropyl starch, carboxymethyl starch, amylose, dextran, chitin, pullulan, gellan gum, xylan, carrageenan, agar, locust bean gum, guar gum, gum arabic, pectin, cellulose, methylcellulose, ethylcellulose, hydroxyethylcellulose, hydroxypropyl-cellulose, ethylhydroxyethylcellulose, hydroxybutylmethylcellulose, hydroxyethylmethylcellulose, oxidized cellulose, carboxymethylcellulose, galactomannan, alginate, chitosan, cyclodextrin, xanthan, hyaluronic acid, heparin, chondroitin sulfate, keratan, dermatan, and polysaccharides having glycosamine residues in natural or diacetylated form and any mixture thereof.

6. The composition according to claim 1, wherein the crosslinker of the crosslinked hydrophilic polymer comprises at least two reactive groups independently selected from the group consisting of hydroxyl group, vinyl group, acrylic group, alkenyl group, alkynyl group, amino group, amido group, carboxylic acid group, ester group and any combination thereof.

7. The composition according to claim 1, wherein the crosslinked hydrophilic polymer has a media uptake ratio (MUR) in the range of 20 to 200.

8. The composition according to claim 1, wherein the crosslinker of the crosslinked hydrophilic polymer is a spacer crosslinker comprising a first optionally substituted aliphatic moiety terminated at each end with a second moiety comprising at least two carboxylic acid groups.

9. The composition according to claim 8, wherein the spacer crosslinker of the crosslinked hydrophilic polymer has the following formula (I):
A-L-Z-L-A (I)
wherein
Z is the first optionally substituted aliphatic moiety;
A is the second moiety comprising at least two carboxylic acid groups; and
L is a linking group.

10. The composition according to claim 8, wherein the first optionally substituted aliphatic moiety is derived from a first optionally substituted aliphatic molecule comprising at least two hydroxy groups, and the second moiety comprising at least two carboxylic acid groups is derived from a second molecule having at least three carboxylic acid groups.

11. The composition according to claim 1, wherein the lipase inhibitor is selected from the group consisting of lipstatin, tetrahydrolipstatin (Orlistat), FL-386, WAY-121898, BAY-N-3176, valilactone, esterastin, ebelactone A, ebelactone B, RHC 80267, cetilistat, and any mixture thereof.

12. The composition according to claim 1, further comprising an amylase inhibitor, a glucosidase inhibitor or any mixture thereof.

13. The composition according to claim 12, wherein the amylase inhibitor and/or the glucosidase inhibitor is selected from the group consisting of acarbose, voglibose, miglitol, emiglitate, camiglibose, pradimicin Q, salbostatin and any mixture thereof.

14. The composition according to claim 12, wherein the composition weight ratio of the crosslinked hydrophilic polymer to the amylase inhibitor and/or the glucosidase inhibitor is greater than 10.

15. The composition according to claim 1, further comprising a pharmaceutically acceptable excipient.

16. The composition according to claim 15, wherein the pharmaceutically acceptable excipient is selected from the group consisting of filler or diluent, disintegrant, coloring agent, lubricant, binder, film-forming agent, wetting agent or emulsifier, and any mixture thereof.

17. The composition according to claim 15, wherein a weight ratio of the excipient to the lipase inhibitor is in the range of 0.01 to 2.

18. A method of forming a composition, comprising the step of contacting a lipase inhibitor with a crosslinked hydrophilic polymer;
wherein the crosslinked hydrophilic polymer has a media uptake ratio (MUR) of at least 20, and an elastic modulus (G′) value in the range of 100 Pa to 10,000 Pa, and
wherein a weight ratio of the crosslinked hydrophilic polymer to the lipase inhibitor is greater than 10.

19. A capsule comprising the composition according to claim 1.

20. A method of treating obesity, pre-diabetes, diabetes, non-alcoholic fatty liver disease (NAFLD), non-alcoholic steatohepatitis (NASH), or chronic idiopathic constipation, or of reducing caloric intake or improving glycemic control in a subject in need thereof, comprising the step of administering to the subject a therapeutically effective amount of the composition according to claim 1.

21. The composition according to claim 1 for use in the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

22. The use of the composition according to claim 1 in the manufacture of a medicament for the treatment of obesity, pre-diabetes, diabetes, non-alcoholic fatty liver disease (NAFLD), non-alcoholic steatohepatitis (NASH), or chronic idiopathic constipation, or for reducing caloric intake or improving glycemic control.

23. The use of claim 22, wherein the composition is administered or is to be administered orally.

24. The use of claim 22, wherein a dosage unit form of the composition that is administered or is to be administered comprises 400 mg to 55,000 mg of the crosslinked hydrophilic polymer, 10 mg to 1000 mg of the lipase inhibitor, optionally 10 mg to 1000 mg of the amylase inhibitor and/or the glucosidase inhibitor and optionally 20 mg to 2000 mg of the pharmaceutically acceptable excipient.
